(19) United States
(12) Patent Application Publication
(10) Pub. No.: US 2022/0178895 A1
(43) Pub. Date: Jun. 9, 2022

(54) GAS MEASURING DEVICE

(71) Applicant: Dräger Safety AG & Co. KGaA, Lübeck (DE)

(72) Inventors: Peter TSCHUNCKY, Lübeck (DE); Doreen GIMBEL, Lübeck (DE); Tim GNOERRLICH, Lübeck (DE); Henrik VEELKEN, Lübeck (DE)

(21) Appl. No.: 17/545,950

(22) Filed: Dec. 8, 2021

(30) Foreign Application Priority Data
Dec. 9, 2020 (DE) 10 2020 132 771.4

(51) Int. Cl. G01N 33/00

(52) U.S. Cl. CPC G01N 33/007; G01N 2033/0072; G01N 33/0006

(57) ABSTRACT

A gas measuring device (100, 400). The gas measuring device (100, 400) includes a chemical gas sensor (103, 417) and a testing unit (101). The testing unit (101) includes a base, a gas duct arrangement arranged at the base with a first gas duct (111, 401) and with at least one additional gas duct (113, 405) and at least one electrochemical gas generator (105, 403, 407). The at least one gas generator (105, 403, 407) is configured to send at least one test gas into the first gas duct (111, 401) in a first state and into the at least one additional gas duct (113, 405) in an additional state. The first gas duct (111, 401) and the at least one additional gas duct (113, 405) are each configured to send at least one gas to the gas sensor (103, 417). The first gas duct (111, 401) differs from the at least one additional gas duct (113, 405).

### CROSS REFERENCE TO RELATED APPLICATIONS

This application claims the benefit of priority under 35 U.S.C. § 119 of German Application 10 2020 132 771.4, filed Dec. 9, 2020, the entire contents of which are incorporated herein by reference.

### TECHNICAL FIELD

The present invention pertains to a gas measuring device, to a tester for a gas measuring device and to a testing process for testing a gas measuring device.

### TECHNICAL BACKGROUND

Gas measuring devices, especially gas warning devices with a gas sensor, must be subjected to function tests at regular intervals in order to ensure their correct functionality.

Limited functionality of the gas measuring device may occur during the operation of a gas measuring device, for example, due to clogging of an opening, through which the gas measuring device is in connection with an ambient medium, or due to mechanical or electrical disturbances.

Processes, in which a test gas is admitted into a gas sensor of the gas measuring device through a gas duct and a corresponding sensor response of the gas sensor is analyzed, are known for detecting a limited functionality of a gas measuring device and for making it possible to repair or to replace the gas measuring device. An effect of disturbance variables cannot be determined accurately in case of all sensor responses based on the use of a single gas duct, as a result of which incorrect measurements may possibly be carried out.

Thus, US 2008/0282765 A1 describes a gas sensor with a single gas duct, with at least one gas sensor, with a gas generator and with a pump. Test gas generated by the gas generator is sent by means of the pump in order to test it.

U.S. Pat. No. 5,667,558 A describes a gas scrubber with an outlet gas sensor and with a pump for feeding chemicals into a scrubbing suspension for scrubbing a gas.

U.S. Pat. No. 4,742,708 A describes an electrochemical gas detection system with an electrochemical sensor and with a housing with a reservoir for an electrolyte. Further, the gas detection system comprises a calibration system with a calibrating gas source for calibrating the gas detection system.

### SUMMARY

An object of the present invention is to provide an improved gas measuring device. The object of the present invention is, in particular, to provide for testing a current functionality of a gas measuring device.

The above object is accomplished by the features according to the invention. Further features and details of the present invention appear from the description and from the drawings. Features and details that are described in connection with the gas measuring device or with the tester are, of course, also valid in connection with the testing process according to the present invention and vice versa, so that reference is and can always mutually be made to the individual aspects of the present invention.

Thus, a gas measuring device is presented.

The gas measuring device comprises a chemical gas sensor configured to react an analyte. The chemical gas sensor includes an enclosing sensor chamber, with at least one opening area, which forms a gas-permeable connection between the sensor chamber and an environment of the gas measuring device. The gas measuring device further comprises at least one testing unit.

The testing unit comprises a gas duct device, particularly a gas duct arrangement, with a number of gas ducts, and at least one gas generator, which is configured to send at least one test gas at a first time period of a test to the testing unit and at a second time period of the test via the gas duct arrangement into the enclosing sensor chamber of the gas sensor. A test gas transport has a different transport characteristic during the first time period than during the second time period based on the duct structure of the corresponding gas duct and/or based on a test gas property of the at least one test gas.

The term “test” is defined in the context of the present invention as a procedure for validating or checking a functionality. It is correspondingly checked during a test whether a predefined function is carried out and/or provided correctly.

A test gas is defined in the context of the present invention as a gas with a known, predefined composition and concentration of substances contained in the test gas.

A duct structure of a gas duct is defined as a structural characteristic of this gas duct, for example, shape, size, cross-sectional area, surface characteristic and/or length of the gas duct.

A gas generator is defined in the context of the present invention as a device comprising at least one generator electrode and a counterelectrode, wherein a common counterelectrode may be provided in case of the use of a plurality of gas generators. The gas generator can generate for this a test gas even with the use of a suitable tablet, such as, e.g., a silver sulfide tablet.

The opening area according to the present invention may be screened, for example, by a filter against coarser particles, but it can nevertheless make possible the passage of ambient medium, especially ambient air. The gas sensor is typically configured for detecting a gas component in the ambient medium entering via the opening area, especially ambient air, outside of the test via the testing unit.

The sensor chamber is a chamber that encloses the gas sensor and is accessible for the ambient medium with the analyte to be analyzed via the at least one opening area. The sensor chamber is preferably defined in space by a sensor housing of the gas measuring device.

A reference value is defined in the context of the present invention as a number of predefined values, which can be put mathematically into a relationship with a comparison value. A reference value may comprise a single value or a plurality of values, especially a curve (relationship). A reference value may comprise positive and/or negative values and may be stated as a range of values. A reference value may be a mathematical processing and/or compression of one or more values.

A computing unit is defined in the context of the present invention as any programmable device. In particular, a computing unit may be a circuit, for example, an ASIC, at least one processor or a distributed system. In particular, a computing unit is a computer.

The gas measuring device being presented comprises at least one chemical gas sensor, which reacts the gas flowing to the gas sensor chemically in order to generate a sensor response, and at least one testing unit. The gas sensor may be a pellistor, i.e., a heat flux sensor, which burns a gas and measures a generated heat flux or an electrochemical sensor, which reduces or oxidizes a gas and measures electrical properties of the reduced or oxidized gas.

The testing unit provided according to the present invention preferably comprises a structure, such as a base, which may consist of a preferably gas-impermeable material, for example, plastic. The base may have especially a round, preferably circular shape and be connected to the gas duct arrangement. The gas duct arrangement may be arranged, for example, in a cross-shaped manner within a circular base.

The testing unit is configured especially to test a functionality of the gas measuring device. The testing unit may test for this purpose a functionality, i.e., a particular function of the gas measuring device, especially of the gas sensor, by a test gas being admitted into the gas sensor by a gas generator.

For admitting gas to the gas generator, the testing unit comprises the gas duct arrangement, with the number of gas ducts, i.e., with at least one gas duct, with a particular duct structure. The test gas is transported via this number of gas ducts in the first time period of the test of the testing unit and in the second time period of the test of the test unit. The two time periods preferably have no overlapping time period or they have only an overlapping time period that is negligible for the measurement. As an alternative, an overlap of the two time periods may be intended for the test according to the present invention by the testing unit.

In addition to the first time period and the second time period, it is also possible according to the present invention to provide at least one additional time period for a test by the testing unit in one embodiment according to the present invention of the gas measuring device.

Due to the different transportation characteristics of the respective gas ducts, a concentration-time profile of a gas to be detected changes at the location of the detection in the gas sensor and therefore so does the sensor response of the gas sensor during the admission of test gas during the two different time periods. The consequence of this is that due to the admission of test gas to the gas sensor during the respective time periods, different gas clouds with different concentration-time profiles develop, which may also be affected differently by ambient parameters, e.g., wind, based on these differences. In particular, a functionality of the gas sensor can be tested by an admission of test gas to the gas sensor during the first time period and a functionality of the opening of the gas measuring device can be tested by an admission of test gas to the gas sensor during the second time period.

Furthermore, sensor values, which were determined by the gas sensor during an admission of test gas to the gas sensor during the first time period, and sensor values that were determined during an admission of test gas to the gas sensor during the second time period can be put with one another into a mathematical relationship in order to infer properties of the gas measuring device or of an environment of the gas measuring device.

In particular, the two transportation characteristics during the two time periods may differ due to different interactions with or different effects of an environment on the gas flowing through the gas duct arrangement. The first gas duct and at least one second gas duct of the gas duct arrangement are preferably constructed for this purpose differently and/or different test gases are used during the two time periods.

In an advantageous embodiment, the gas duct arrangement has at least two gas ducts, wherein at least one test gas is sent via a first gas duct to the sensor chamber during the first time period and is sent via a second gas duct to the sensor chamber during the second time period. As a result, the location of the test gas transportation during the first time period differs from the location of the test gas transportation during the second time period. The gas duct arrangement may also have, in principle, at least one additional gas duct in addition to the first gas duct and to the second gas duct.

In an advantageous variant of the above embodiment, the first gas duct differs from the second gas duct in terms of its duct structure.

Provisions may be made for a first gas duct opening of the first gas duct to be located closer to the gas sensor than does a second gas duct opening of the second gas duct.

As an alternative or in addition, provisions may be made for the second gas duct opening to be located closer to the at least one opening area than does the first gas duct opening.

Test gases with different flow properties and thereby with a different transportation characteristics can be generated for admission to the gas sensor by means of a different duct structure of the respective gas ducts of the gas measuring device being presented. For example, a first gas duct, which has a gas duct geometry that ends at a short distance in front of the gas sensor, can be used for testing the gas sensor per se, while a gas duct that has a gas duct geometry that ends at a greater distance from the gas sensor relative to the first gas duct can be used to test a flow characteristic of the opening area and/or of a sensor inlet of the gas measuring device.

In case of a short distance between an end of a gas duct and a gas sensor, environmental effects, for example, bypass flows caused by ambient wind, have a minimal effect on corresponding sensor values. Such a gas duct geometry is correspondingly especially advantageous for testing the operability and the value of the sensitivity of the gas sensor, because a low disturbance variance and/or a weak effect of disturbance variables can be expected during a corresponding test.

Since environmental effects, for example, bypass flows caused by ambient wind, have a strong effect on corresponding sensor values in case of a great distance between an end of a gas duct and a gas sensor, and an opening area and/or a sensor inlet of the gas measuring device is preferably located closer to the end of the gas duct than does the gas sensor, such a gas duct geometry is especially advantageously suitable for testing an opening area and/or a sensor inlet of the gas measuring device. Such a gas duct geometry is especially suitable in an especially advantageous manner for testing an opening area and/or a sensor inlet of the gas measuring device, because a degree of clogging of an opening has an especially great effect on the transportation characteristics of the corresponding test gas between the end of the gas duct and the gas sensor.

Provisions may further be made for the first gas duct and the at least one additional gas duct to differ from one another by a roughness of their inner surface in their duct structures.

Test gases with different transportation characteristics, especially with different flow properties, can be generated for admitting gas to the gas sensor by means of different roughnesses of inner areas of the respective gas ducts of the gas measuring device being presented. For example, a first gas duct, whose inner surface has a low roughness, i.e., is especially smooth, can be used for testing the gas sensor per se, while a gas duct, which has an inner surface with a greater roughness relative to the first gas duct, can be used to test a flow characteristic of the opening area of the gas measuring device.

Since a test gas flowing through the gas duct is flowing at a very high velocity in case of a low roughness of an inner surface of a gas duct, environmental effects, for example, bypass flows caused by ambient wind, affect the compact gas cloud at the outlet hole of the gas duct to a lesser extent than they would in case of a decelerated, diffuse release of gas in the outlet area of a rough duct. Such a gas duct is correspondingly especially advantageously suitable for testing the operability and the value of the sensitivity of the gas sensor, because a low disturbance variance and hence a weaker effect of disturbance variables can be expected during a corresponding test.

Since a test gas flowing through the gas duct flows more slowly based, for example, on adsorption and desorption processes at the walls in case of a great roughness of an inner surface of a gas duct, the development of a gas cloud at the outlet is delayed in time, it takes place over a longer time period and with a lower concentration at the location of detection and it is therefore affected differently by environmental effects, for example, wind. Such a gas duct is correspondingly especially advantageously suitable for testing an opening of the gas measuring device, because the degree of clogging of such an opening is linked mathematically and is thereby correlated with an effect of bypass flows caused by ambient wind.

Provisions may further be made for the at least one gas duct to comprise a temperature control unit, which is configured to change a temperature in the interior of the gas duct such that the gas temperature of the test gas differs in the first time period from that of the test gas in the second time period. The test gas may thus be different in the first time period from the test gas in the second time period.

Test gases with different transportation characteristics can be generated for admitting gas to the gas sensor by means of a different temperature control of the test gas of the gas measuring device being presented. For example, a first gas duct with a temperature control unit may be used for heating the first gas duct for testing the gas sensor, while another gas duct of the gas duct arrangement, which gas duct has no temperature control unit or has a temperature control unit for cooling the gas duct, can be used to test a flow characteristic of the opening of the gas measuring device.

Since if a gas duct has a high temperature, a test gas flowing through the gas duct flows at a high velocity, the test gas leaves the gas duct as a compact gas cloud and ambient atmospheric effects, for example, wind, have a weaker effect on the compact gas cloud at the outlet hole of the gas duct than they would have at a lower temperature of the gas duct. A heated and/or heatable gas duct is especially advantageously suitable for testing the gas sensor, because a low disturbance variance, i.e., a weak effect of disturbance variables, can be expected during a corresponding test.

Since a test gas flowing through the gas duct flows slowly in case of a low temperature of a gas duct, a gas cloud developing with a time delay is formed over a longer time period at the outlet and it has a lower concentration at the detection location and it is therefore affected and/or can be affected differently by environmental effects, for example, wind. A cooled and/or coolable gas duct is correspondingly especially advantageously suitable for testing an opening of the gas measuring device against the ambient atmosphere, because the degree of clogging of such an opening is linked mathematically and thereby correlated with an effect of bypass flows caused by ambient wind.

Provisions may further be made for the first gas duct and for the at least one additional gas duct to be in fluid connection with one another especially via respective chambers of the gas ducts and for the first gas duct not to be in direct fluid contact with an environment of the testing unit and for the at least one additional gas duct to be in direct fluid contact with the environment of the testing unit.

A gas duct with a chamber, which is not in direct fluid contact with an environment, is especially advantageously suitable for minimizing the effect of environmental effects on a test gas for testing a gas sensor.

A gas duct with a chamber that is in direct fluid contact with an environment is especially advantageously suitable for maximizing the effect of environmental effects on a test gas for testing a gas sensor.

In a preferred embodiment, the at least one gas generator is configured to send a different test gas through the corresponding gas duct during the first time period than during the second time period. The two different test gases especially preferably have two different diffusion coefficients. As a result, different transportation characteristics, especially different concentration-vs.-time profiles, are made possible regardless of the structure of the gas duct arrangement.

In one embodiment, there is a time interval, during which no test gas is provided by the gas generator between the first time period and the second time period, and this time interval is at least 1 minute, especially at least 30 minutes, and especially preferably at least 2 hours. There is a clear separation in time in this embodiment between a first phase of the test during the first time period and a second phase of the test during the second time period. As a result, chemical processes, which take place during the first time period, can be allowed especially reliably to take place before another phase of the testing of the gas measuring device takes place during the second time period.

The testing unit of the gas measuring device being presented may comprise at least one computing unit. The at least one computing unit is preferably configured to receive a signal of the gas sensor and to determine therefrom a measured value, wherein the computing unit is further configured to determine and to output test information during the first time period and/or during the second time period, wherein the test information indicates whether the gas measuring device is defective, and wherein the determination of the test information is based on a comparison between the measured value and a predefined gas sensor threshold value or between a defined measured value curve (measured value relationship) parameter and a predefined curve (relationship) parameter. The test information may indicate here, for example, whether the at least one opening area is clogged and/or whether the gas sensor is operable. Different examples are shown in the following embodiments for the possible manner of operation of such a computing unit. The use of a comparison between predefined measured values or a predefined measured value curve makes possible an especially simple and reliable determination of the test information by the computing unit.

The computing unit is configured in an example of the above embodiment to carry out the comparison between the measured value and a predefined gas sensor threshold value for one of the two time periods and to carry out the comparison between the measured value curve parameter and the predefined curve parameter for the other of the two time periods. Different determination instructions, which lead ultimately to the test information, are carried out hereby during the respective time periods.

As an alternative or in addition, an analysis may be carried out by the computing unit over a plurality of past measured values from first and/or second time periods. The fact that the history of measured values is taken into consideration in such a manner can lead to an especially reliable analysis.

As an alternative or in addition, the computing unit may comprise a control module for controlling components of the gas measuring device, for example, the gas generator. As an alternative or in addition, the at least one computing unit may comprise an analysis module for analyzing respective measured values of the gas sensor of the gas measuring device being presented. In particular, the at least one computing unit is used to operate the gas measuring device being presented. The at least one computing unit can be connected for this purpose to the gas sensor and/or to respective gas generators for communication in order to exchange control commands with the gas sensor and/or with the respective gas generators. The at least one computing unit may comprise one or more processors, which are configured to operate the gas measuring device being presented. The at least one computing unit may be located in a computer, for example, a server, especially a cloud, and be in communicative connection with the gas measuring device being presented via a communication interface. As an alternative, the at least one computing unit may be located at least partially in the gas measuring device itself.

Provisions may further be made for the computing unit provided according to the present invention to be configured to mark the gas sensor or the gas measuring device as being defective in case a difference or a quotient of measured values to be determined by the gas sensor during the first time period or interim results that can be calculated therefrom and at least one predefined reference value is greater than a predefined gas sensor threshold value, and to mark the opening area, through which the gas duct is in direct connection with the environment, as being clogged in case a difference or a quotient of a variable describing the signal value, such as of the decay time, of the peak height, of the integral or interim results of measured values determined by the gas sensor during the second time period, which interim results can be calculated therefrom (from the integral), is greater than a predefined clogging threshold value.

A functionality of the gas sensor can be tested by means of a mathematical comparison measured value determined during an admission of test gas through the gas duct provided according to the present invention and/or of a value obtained by the further mathematical processing of these measured values and a predefined reference value.

Since the gas sensor provided according to the present invention is a chemical gas sensor, it reacts the test gas chemically in case of correct, i.e., error-free functionality, so that a concentration of the test gas decreases over time and measured values determined by the gas sensor change correspondingly.

Should the comparison between respective measured values of the gas sensor, which are determined during an admission of gas during the first time period, and a reference value show no change or only a slight change in the measured values, because, for example, a difference or a quotient between the measured values and/or between a value obtained by the further mathematical processing of the measured values and the reference value is above a predefined gas sensor threshold value, defective functionality of the gas sensor or of the gas measuring device can be inferred. The computing unit provided according to the present invention is correspondingly configured to mark in such a case the gas sensor or the gas measuring device as being defective and to store, for example, an error message in a memory of the gas sensor, of the gas measuring device and/or in an error memory and/or in a memory of the computing unit. The reference value may be in this case, for example, a sensor value at a half-time of a decay curve of a reference sensor or pertain to the change of the parameter of the respective sensor itself.

A functionality of the opening of the gas measuring device can be tested by means of a mathematical comparison of a parameter determined during an admission of test gas during the second time period, for example, a decay time, of a peak height, of an integral or of additional characteristics and a predefined reference value. Since bypass flows, for example, ambient air, flow into the gas measuring device and/or test gas flows out of the gas measuring device, a concentration of a test gas is minimized over time in case of an opening that is not clogged, so that a concentration of the test gas drops over time and measured values determined by the gas sensor change correspondingly rapidly, a parameter, which describes the height and the size of the signal, is especially advantageous for testing a functionality of the opening. Such a parameter would be, for example, the peak height in combination with the decay time or the reacted quantity of gas at the sensor, which quantity can be plotted over the integral.

To check the proper sensitivity of a gas sensor, test gas can be admitted into the gas sensor by means of the gas generator according to the present invention. The gas admission takes place here at first such that a, for example, first gas duct, which is independent from respective ambient conditions as much as possible and is not directly in contact with an environment, is used.

To analyze whether an unhindered entry of gas to a gas sensor to be tested can take place and/or to determine possible blockages or clogging, test gas is sent by means of the gas generator according to the present invention, for example, through a second gas duct to the gas sensor, which promotes an especially strong interaction of the test gas with the ambient atmosphere and is preferably in direct contact with or is separated only by a filter from an environment.

As a consequence of the feed of gas by the gas generator provided according to the present invention, the gas sensor reacts with a sensor signal, which is proportional to the concentration of the test gas fed, both in case of an admission of gas through the first gas duct and in case of admission of gas through the second gas duct. A time curve (time relationship or time course) of measured values determined by the gas sensor, which time curve develops in the process, is analyzed by an analysis unit for a gas admission through the first gas duct and for a gas admission through the second gas duct.

On the one hand, it is possible at the time of the analysis of the measured values of the gas duct to carry out a comparison between a general curve shape of a concentration-time profile of the gas sensor and a predefined reference value, which is stored, for example, in a memory of the analysis unit, in the form of a fit function. It is possible by such a comparison to obtain a qualitative analysis of the curve shape of the measured values determined by the gas sensor. Such a comparison with a fit function forms a kinetics of a measured signal of the gas sensor against the time on the basis of a standardized curve or of a curve in, for example, concentration units. Especially suitable fit parameters of a mathematical function can be used for this.

Mathematical parameters of the measured values, such as maximum, minimum, half-value width, half-time of a dropping branch of the measured values, a signal height of the measured values, for example, in concentration units at defined times on the dropping branch, area integrals with different limits, standard deviations over different value ranges, medians as well as any further, technically suitable parameter may be used for the quantitative analysis of the measured values of the gas duct.

In particular, the comparison carried out by the computing unit to determine the test information preferably depends on a determination of a decay time, which determination is based on the determined measured value curve, of a mathematical deviation, of a statistical mean, of a maximum and/or of an integral as well as of an examination of earlier measured values.

Furthermore, it is possible to derive characteristic variables, which sufficiently describe a behavior of the gas sensor, by extrapolation from partial segments of the measured values. For example, information on a response characteristic, response times and dead times of the sensor can be determined in a plotting of measured values in concentration units against the time from a linear extrapolation over a selected value range between two times before a maximum is reached. Information on a decay characteristic of the sensor and on the absence of clogging of a gas access of the gas sensor can correspondingly be determined after the maximum has been reached. Further suitable plottings of the measured values of the gas sensor can be used to analyze the functionality of the gas sensor, For example, the measured values of the gas sensor can be plotted and analyzed against a gas dose, i.e., an integral over a time curve of the measured values, in order to determine the test information.

Determined actual values, i.e., measured values of the gas sensor and reference values, can, in particular, be compared with one another by suitable mathematical methods. The reference values may be predefined, for example, as tolerance ranges, which describe ranges for properly working gas sensors and gas generators. Different states or malfunctions of the gas sensor can be inferred from deviations or overshooting of the measured values of the gas sensor from these tolerance ranges. For example, the gas sensor may respond to the test gas too insensitively, too sensitively, too slowly and too rapidly, and the test gas may be removed and/or processed further too rapidly or too slowly. In particular, a curve shape of the measured values may be shifted in time or in terms of its concentration values compared to a predefined reference curve. Any deviation from a predefined reference value is analyzed corresponding to an analysis logic stored in an analysis unit and is optionally outputted to an end user after a repeated test has been carried out. In particular, a handling recommendation can be outputted for a user, especially within the framework of the test information on the basis of an analysis of the measured values of the gas sensor.

Should the comparison between the determined decay time and the reference value show a slow change of the measured values because, for example, a difference between the decay time and the reference value is above a predefined gas sensor threshold value, a defective functionality of the opening and/or closing can be inferred and the gas measuring device is correspondingly marked as being defective. The computing unit provided according to the present invention is correspondingly configured to mark in such a case the opening, especially the opening area, or the gas measuring device as being defective and to store, for example, an error message in a memory of the gas measuring device and/or in an error memory and/or in a memory of the computing unit. The reference value may be in this case, for example, a determined sensor value at a decay time, especially at a half-time of a decay curve of a reference sensor in a reference chamber with a fully patent opening.

Provisions may further be made for the computing unit to be configured to mark the gas measuring device by corresponding test information as being defective in case measured values are higher or lower than a predefined reference value after a predefined time in case of admission of test gas to the gas sensor through a gas duct whose inner surface has a roughness that is lower than the roughness of an inner surface of a respective other gas duct and to mark as being clogged an opening through which the respective other gas duct is in direct fluid contact with an environment of the gas measuring device in case measured values that are higher or lower after a predefined time than a predefined reference value in case of admission of test gas to the gas sensor through the respective other gas duct.

Since a reaction of a test gas is interfered with in case of a defective gas sensor and the concentration of the test gas changes, as a result of this, only minimally, a functionality of the gas sensor can be inferred and the corresponding test information can as a result be outputted in case of measured values that are determined at a time at which a reaction of the test gas at a reference sensor affects the concentration of the test gas. The measured values of the gas sensor can be analyzed at this time for this purpose, so that it can be assumed that the gas sensor and correspondingly the gas measuring device are defective in case these measured values are higher than a predefined reference value or the measured values differ from a maximum of the concentration of the test gas only by a value that is below or, depending on the sign of a corresponding measured signal, above a predefined threshold value.

Since disturbance variables are to be minimized during a test of the gas sensor, admission of gas through a gas duct suitable for an especially rapid transportation of gas, especially through a gas duct with an especially smooth inner surface, is especially advantageously suitable for testing the gas sensor.

Since an interaction between a test gas and an environment is considered in a test of the opening of the gas measuring device, admission of gas to the gas sensor through a gas duct that causes the test gas to be able to interact with an environment for an especially long time is suitable. Especially a gas duct with an especially rough inner surface may be suitable for this purpose. The test gas is slowed down through a gas duct with a rough inner surface and it correspondingly flows slowly and, as a result of this, over a long time to the gas sensor for the interaction with an ambient medium.

Provisions may further be made for the computing unit to be configured to mark the gas sensor and/or the gas measuring device as being defective based on the test information in case measured values are higher or, depending on the sign of a corresponding measured signal, lower than a predefined reference value after a predefined time in case of admission of test gas to the gas sensor through a gas duct whose inner temperature is higher than an inner temperature of a respective other gas duct, and to mark as clogged an opening through which the respective other gas duct is in direct fluid contact with an environment of the gas measuring device in case measured values that are higher or correspondingly lower than a predefined reference value after a predefined time in case of admission of test gas to the gas sensor through the respective other gas duct.

Since disturbance variables are to be minimized in a test of the gas sensor, admission of gas through a gas duct suitable for an especially rapid transportation of gas, especially through an especially hot gas duct, is especially advantageously suitable for testing the gas sensor.

Since an interaction between a test gas and an environment is considered in a testing of the opening of the gas measuring device, admission of gas to the gas sensor through a gas duct suitable for an especially slow transportation of gas, especially through an especially cold gas duct, is especially advantageously suitable for testing the opening.

Provisions may further be made for the computing unit to be configured to recalibrate the gas sensor on the basis of the difference in case a difference of measured values to be determined by the gas sensor during at least one first state and at least one predefined reference value is greater or lower than a predefined gas sensor threshold value.

The gas sensor can be recalibrated in order to repair a gas sensor marked as being defective or to continuously adapt the gas sensor to a current situation. Provisions may be made for this purpose, for example, for a comparison table or an assignment logic to assign respective measured values determined by the gas sensor to respective values of an output scale, which values are to be outputted, as a function of a deviation between measured values determined during the first state and a predefined reference value. For example, a warning threshold, starting from which a warning sound is to be outputted, is raised or lowered for this as a function of the deviation. A warning sound is also test information in this sense. In general, test information is information that indicates a result of the test during at least one of the two time periods according to the present invention.

Provisions may be made, in particular, for using a trend of measured values determined by the gas sensor in order to recalibrate the gas sensor. For example, a mean value or each further, technically suitable characteristic of a plurality of measured values determined staggered over time may be formed for this purpose.

Provisions may further be made for the first gas duct to be configured such that the test gas flows faster through the first gas duct than through the second gas duct and for the computing unit to be configured to determine a difference of a first time between a start of an admission of test gas through the first gas duct and a time of an increase of the measured values of the gas sensor as well as another time between a start of an admission of test gas through the at least one additional gas duct and a time of an increase of measured values of the gas sensor and to infer a diffusion time of the test gas to the measuring device on the basis of the difference and to take this diffusion time into consideration when carrying out a test.

By determining such a diffusion time, which can change preferably as a function of an ambient temperature, it is possible to correct variables to be determined by the computing unit of the gas measuring device, for example, a decay time or each additional variable to be calculated or to be determined, for example, a temperature.

Provisions may further be made for the computing unit to be configured to infer a wind speed outside of the opening area on the basis of a comparison of measured values to be determined by the gas sensor during the first time period and measured values to be determined by the gas sensor during the second time period.

Properties of a disturbance variable interacting with the test gas, especially a velocity of a bypass flow flowing in through the opening, can be inferred by a comparison of measured values of the gas sensor which were obtained during the admission of test gas which was obtained by the use of a gas duct carrying gas at a high velocity, with measured values of the gas sensor which were obtained during the admission of test gas by means of a gas duct carrying gas at a low velocity. A wind speed outside of the gas measuring device and consequently outside of the opening area can correspondingly be inferred on the basis of the comparison. The greater is the difference of occurring deviations from reference values, the higher is the wind speed.

Provisions may further be made for the testing unit to comprise at least a first gas generator and at least one additional gas generator, and the computing unit is configured to admit test gas to the gas sensor by the at least one additional gas generator in case the gas sensor is to be marked as being defective during an admission of gas by the first gas generator, to mark the gas sensor as being error-free and the first gas generator as being defective and to provide the corresponding test information in case the gas sensor is not to be marked as defective in case of admission of test gas by the additional gas generator.

For example, a validation generator, which is preferably used only if a gas sensor is to be marked as being defective, can be used by means of a plurality of gas generators to check the gas generator that is subject to a test that has led to the result that the gas sensor or the opening is to be marked as being defective. A falsely defective marking of a gas sensor or gas measuring device can correspondingly be avoided by the validation generator.

Provisions may further be made for the first gas duct and for the second gas duct to be arranged parallel next to one another or to be connected into at least one labyrinth.

Environmental effects on respective measured values to be determined by means of the gas sensor can be minimized by a labyrinth of gas ducts.

In a second aspect, the invention being presented pertains to a tester for a gas measuring device. The tester comprises a possible embodiment of the testing unit provided according to the present invention and an interface (connection interface) for a reversible mechanical and/or communicative connection (an operative connection interface that can be connected, disconnected and reconnected) of the testing unit to a gas sensor.

The tester according to the present invention can preferably be connected reversibly to a gas measuring device and/or to a gas sensor, so that the tester can be used for testing a plurality of gas measuring devices. Reference is correspondingly made concerning the tester to the advantages described within the framework of the gas measuring device being presented.

In a third aspect, the invention being presented pertains to a testing process for testing a gas measuring device. The testing process comprises the following steps:
a) provision of a gas measuring device according to one of the above embodiments;
b) actuation of the at least one gas measuring device such that this sends at least one test gas during a first time period of a test and during a second time period of the test via the gas duct device (a gas duct arrangement) into the enclosing sensor chamber of the gas sensor;
c) determination of measured values determined by the gas sensor during the first time period and/or during the second time period; and
d) determination and output of test information during the first time period and/or during the second time period, wherein the test information indicates whether the gas measuring device is defective, and wherein the determination of the test information is based on a comparison between the measured value or of a parameter calculated therefrom and a predefined gas sensor threshold value or between a defined measured value curve parameter calculated according to a predefined instruction and a predefined curve parameter.

The testing process being presented is used especially to operate the gas measuring device being presented as well as the tester being presented, so that reference is made to the described advantages of the gas measuring device concerning the advantages of the process being presented.

Further measures improving the present invention appear from the following description of some exemplary embodiments of the present invention, which are shown in the figures. All the features and/or advantages appearing from the claims, from the description or from the drawings, including structural details and arrangements in space, may be essential for the present invention both in themselves and in the different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic cut-away side view of an embodiment of the gas measuring device according to the present invention;

FIG. 2 is a comparison of measured data, which were determined with the use of the gas measuring device from FIG. 1 during the admission of gas through a first gas duct and measured data that were determined during an admission of gas through a second gas duct;

FIG. 3 is a comparison of measured data that can be used to determine the effect of an ambient velocity with the use of the gas measuring device from FIG. 1;

FIG. 4 is another possible embodiment of the gas measuring device of the present invention; and

FIG. 5 is flow diagram showing the course of a possible embodiment of the process according to the present invention.

### DESCRIPTION OF PREFERRED EMBODIMENTS

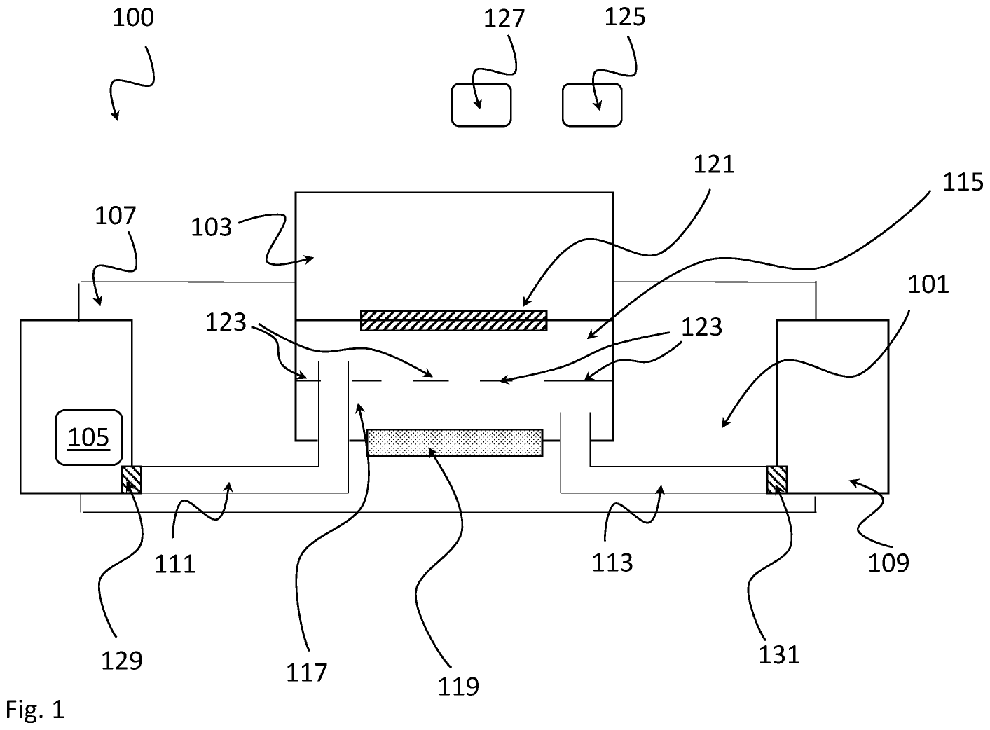

Referring to the drawings, FIG. 1 shows a gas measuring device 100 in a cut-away side view. The gas measuring device comprises a testing unit 101 and a gas sensor 103.

The testing unit 101 comprises an especially chemical gas generator 105 for reacting an analyte of a test gas for testing the gas measuring device 100.

The gas generator 105 comprises a generator electrode in a first generator chamber 107 and a counterelectrode. Furthermore, an additional generator chamber 109 is provided. Provisions may be made, in particular, for the counterelectrode to be used jointly by a plurality of gas generators/generator chambers.

The first generator chamber 107 is in contact with a first gas duct 111 via a first gas discharge opening 129, so that test gas generated by the gas generator 105 flows through the first gas discharge opening 129 and into the first gas duct 111. In order to prevent a discharge of electrolyte possibly being stored in the first generator chamber 107 into the first gas duct 111, the first gas discharge opening 129 comprises a gas-permeable diaphragm, which is permeable for the test gas and impermeable for the test gas.

The additional generator chamber 109 is in contact with a second gas duct 113 via an additional gas discharge opening 131, so that test gas generated by the optional additional gas generator and/or by an optional additional generator electrode flows through the additional gas discharge opening 131 into the second gas duct 113, to prevent electrolyte possibly being stored in the additional generator chamber 109, the additional gas discharge opening 131 comprises a selectively gas-permeable diaphragm, which is permeable for the test gas and impermeable for the electrolyte.

The testing unit 101 is connected in this case permanently to the gas sensor 103. In addition, the testing unit 101 is configured such that the testing unit 101 has an interface for communicative and/or mechanical connection to the gas sensor 103.

The testing unit 101 may be configured as a circular base, at which the first gas duct 11 and the second gas duct 113 are arranged or into which the first gas duct 111 and the second gas duct 113 are integrated.

The first gas duct 111 further comprises a first chamber 115.

The second gas duct 113 further comprises an additional chamber 117 with an opening area 119, through which the chamber 117 is in contact with an environment via a filter at the opening area 119, so that ambient medium, e.g., air, can flow into the chamber 117 and test gas can flow out of the chamber 117. The first chamber 115 and the additional chamber 117 are fluidally in exchange with one another, so that gases can flow from the additional chamber 117 into the first chamber 115. The flow motion between the first chamber 115 and the additional chamber 117 is led through and/or defined by flow guiding devices 123. The first chamber 115 and the additional chamber 117 form a sensor chamber enclosing the gas sensor 103 in the exemplary embodiment shown.

The first gas duct 111 has a duct structure that differs from the duct structure of the second gas duct 113. The first gas duct 111 is longer in this case than the second gas duct 113, so that a distance between an end of the first gas duct 111 and an inlet area 121 of the gas sensor 103 is shorter than a distance between an end of the second gas duct 113 and the inlet area 121 of the gas sensor 103.

Furthermore, the testing unit 101 comprises a first computing unit 125, which is configured as a control module to control the gas generator 105 and the gas sensor 103. The first computing unit 125 can be in connection for this purpose with the gas generator 105 and with the gas sensor 103 via a communication interface, such as an operative connection interface that can be connected, disconnected and reconnected, for example, a cable or a wireless connection. The first computing unit 125 may comprise one or more processors, which are configured for controlling the gas generator 105 and the gas sensor each individually or jointly.

For testing the gas measuring device 100, the gas generator 105 is actuated by the first computing unit 125 such that in a first state, during a first time period of a test of the testing unit 101, the gas generator 105 admits test gas into the gas sensor 103 by means of the first gas duct 111. In a second state, during a second time period of the test, the first computing unit 125 actuates the gas generator 105 such that this admits test gas into the gas sensor 103 by means of the second gas duct 113.

Since the first gas duct 111 and the second gas duct 113 differ from one another in terms of their duct structures, the test gas flows with different transportation characteristics through the first gas duct 111 and through the second gas duct 113.

The test gas is discharged in this case during the first time period at the end of the first gas duct 111 at a short distance from the inlet area 121 of the gas sensor 103. The test gas correspondingly flows rapidly, i.e., directly to the gas sensor 103, as a result of which an effect of disturbance variables, for example, inflows of an ambient medium from the environment and outflows of test gas into the environment, is minimal A measurement of the test gas by the gas sensor 103 is therefore subject during the first time period to an effect of disturbance variables that is reduced relative to the second time period. Measured values determined by the gas sensor 103 during the first state are correspondingly especially valid concerning the functionality of the gas sensor 103.

In the additional state during the second time period, the test gas is discharged at the end of the second gas duct 113 at a great distance from the sensor inlet 121 and in the proximity in space of the opening area 119.

Based on the long diffusion paths combined with a long diffusion time, measured values determined by the gas sensor 103 during this second time period are especially susceptible to disturbing effects caused by the inflow of an ambient medium and/or by an outflow of test gas. The outflow of the test gas, in particular, represents an important removal path of the test gas generated, because the outlet opening of 113 is located in the immediate proximity of the opening area 119 with the gas inlet opening. Correspondingly, the measured values determined during the second time period are especially affected by and are valid concerning the detection of a clogging of the opening area 119.

Provisions are correspondingly made for the functionality of the gas sensor 103 to be tested on the basis of measured values determined during the first time period and for the functionality or the permeability of the opening area 119 to gases to be tested on the basis of measured values determined during the second time period.

In addition to the first computing unit 125, a second computing unit 127, which is configured as an analysis module and is in communicative connection with the gas sensor 103 via a communication interface, for example, a cable or a wireless interface, may optionally also be used for the analysis of measured values determined by the gas sensor. The first computing unit 125 may, of course, also be used for the analysis of measured values determined by the gas sensor 103.

In one exemplary embodiment, not shown, a different test gas is sent through the corresponding gas duct, i.e., for example, through the first gas duct, during the first time period than during the second time period. The two different test gases preferably differ from one another in their diffusion coefficients, so that the transportation characteristic is different during the first time period from that seen during the second time period already due to the different test gases.

Only a single gas duct is provided in another exemplary embodiment to provide the two time ranges with different transportation characteristics of the test gas transportation. The different transportation characteristics are based in this case on different test gas properties, for example, on different test gases used and/or on different test gas temperatures used.

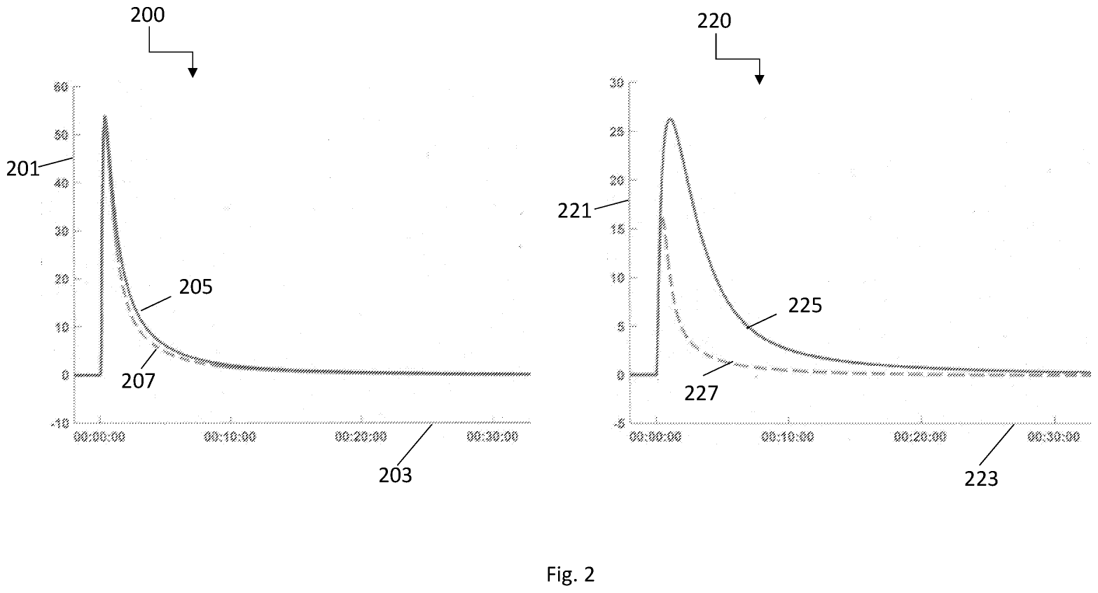

FIG. 2 shows a diagram 200, having an ordinate 201 indicating a sensor signal in [ppm] and having an abscissa 203 indicating a time in [hh:mm:ss].

A curve (time relationship or time course) 205 represents measured values that were determined by the gas sensor 103 during an admission of test gas in the first state during the first time period from the first gas duct 111 and while the opening area 119 was clogged for gases, i.e., impermeable to gases.

A curve (time relationship or time course) 207 represents measured values that were determined by the gas sensor 103 during an admission of test gas in the first state during the first time period from the first gas duct 111 and while the opening area 119 was permeable to gases, i.e., while opening area 119 was not clogged.

Furthermore, FIG. 2 shows a diagram 220, which shows a sensor signal in [ppm] on its ordinate 221 and the time in [hh:mm:ss] on its abscissa 223.

A curve 225 represents measured values that were determined during the admission of test gas in the additional state during the second time period from the second gas duct 113 and while the opening area 119 was clogged for gases, i.e., impermeable to gases.

A curve 227 shows measured values that were determined by the gas sensor 103 during an admission of test gas in the additional state during the second time period from the second gas duct 113 and while the opening area 119 was permeable to gases, i.e., while opening area 119 was not clogged.

A comparison of the curves 205 and 207 with the curves 225 and 227 shows that the clogging of the opening area 119 during the admission of test gas through the second gas duct 113 according to the curve 227 differs markedly from the curve 225. Furthermore, the clogging of the opening area 119 during the admission of test gas through the first gas duct 111 according to the curve 205 differs very slightly from the curve 207. Correspondingly, a distinction can clearly be made on the basis of measured values determined by the gas sensor 103 during the additional state between a state in which the opening area 119 is clogged and a state in which the opening is permeable to gases.

Measured values determined by the gas sensor 103 during the second time period can therefore be used to detect a clogged state in order to determine, i.e., to calculate or to estimate a decay time, for example, a half-time or another value at a predefined time after the start of the admission of gas. Should a difference in the decay time from a predefined reference value be greater than a predefined clogging threshold value, it can be assumed that the opening area 119, especially a filter within the opening area 119, is clogged. Provisions are correspondingly made for this case for the computing unit 125 to mark the gas measuring device 100 as being clogged and, for example, to store a corresponding error message in an error memory.

A decay time can be calculated, in particular, for example, by an area under a maximum between a predefined start time and a predefined stop time.

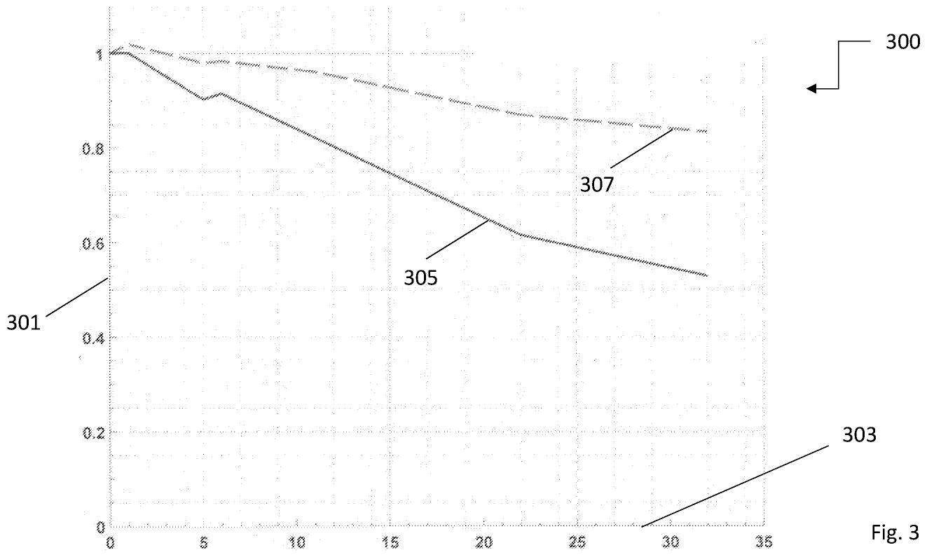

FIG. 3 shows a diagram 300, which shows a unitless relative sensor signal on its ordinate 301. This sensor signal changes due to the effect of the wind for different wind speeds, which were plotted on the abscissa 303 in [m/sec].

A curve 305 is based on measured values that were determined by the gas sensor 103 during the admission of test gas through the second gas duct 113.

A curve 307 is based on measured values that were determined by the gas sensor 103 during the admission of test gas through the first gas duct 111.

It can be clearly seen that a distance between the curve 305 and the curve 307 increases with increasing wind speed. This means that a corresponding wind speed can be inferred from a distance between the curve 305 and the curve 307 and that the device used for the curve 307 is markedly less subject to the effect of wind and is therefore better suited for checking the sensitivity of the sensor 301. This relationship may be used, for example, to deactivate a warning function of the gas measuring device 100 when wind speeds that are higher than a predefined threshold value are determined.

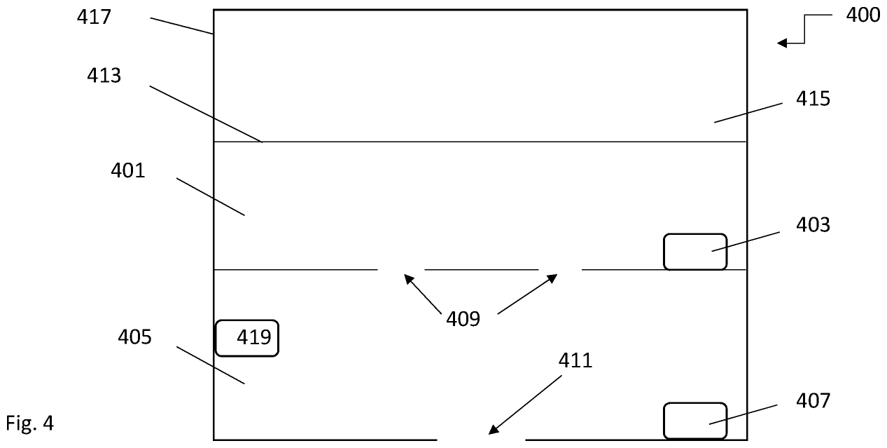

FIG. 4 shows a gas measuring device 400.

The gas measuring device 400 comprises a first gas duct 401, which is configured here as a chamber and in which a first gas generator 403 is arranged, and a computing unit 419.

The gas measuring device 400 comprises, furthermore, a second gas duct 405, which is configured here as a chamber and in which an additional gas generator 407 is arranged.

The first gas duct 401 is in fluid contact with the second gas duct 405 via gas transfer openings 409, so that an exchange of gas between the first gas duct 401 and the second gas duct 405 is possible.

The second gas duct 405 is in fluid contact with an environment via an opening 411, which acts as a sensor inlet and forms in this sense the opening area for the gas measuring device 400, so that a direct, immediate gas exchange is possible between the environment and the second gas duct 405.

The first gas duct 401 is in contact with a sensor diaphragm 413, which separates an electrolyte of a gas sensor 417 of the gas measuring device from the first gas duct 401.

Since the first gas duct 401 is not directly in contact with an environment, a test gas provided by the first gas generator 403 is affected only minimally by ambient conditions of the gas measuring device 400, for example, by wind. The first gas duct 401 is correspondingly especially advantageously suitable for testing the gas sensor 417.

In order to rule out a false negative error message, which falsely marks the gas sensor 417 as being defective in case measured values of the gas sensor 417 show upon the admission of test gas to the interior space 415 of a sensor by the first gas generator 403 that the gas sensor 417 is to be marked as being defective, the first gas generator 403 can be tested by means of the additional gas generator 407.

To test the first gas generator 403, provisions are made for a test gas provided by the additional gas generator 407 to be admitted to the gas sensor 417. In case the gas sensor 417 is not to be marked as being defective upon admission of test gas by the additional gas generator 407, provisions are made for the gas sensor 417 to be marked as error-free and the first gas generator 403 as being defective. A computing unit of the gas measuring device 400 can change or generate for this purpose a corresponding error message in an error memory of the gas measuring device 400 or in a memory of the computing unit.

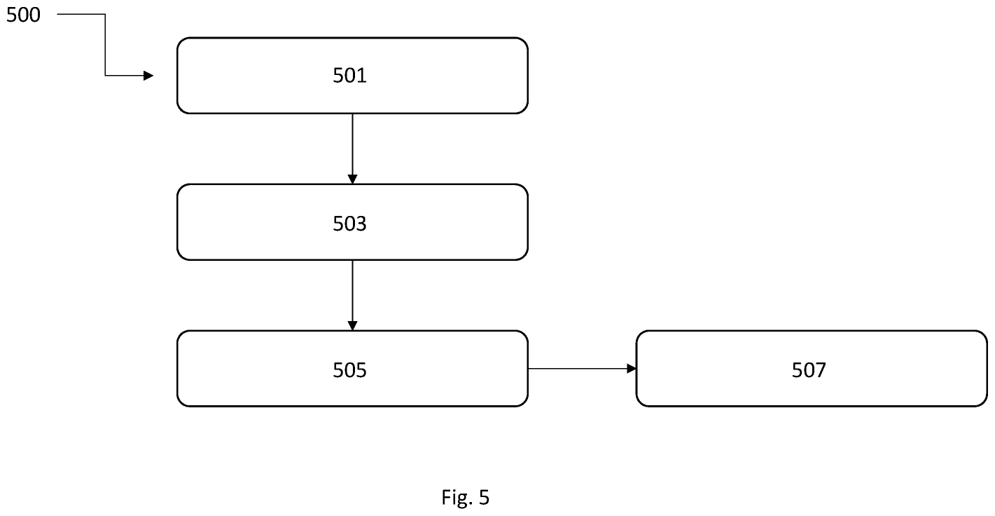

FIG. 5 shows the course of the process 500 described.

The process 500 starts with a provision step 501 for providing a possible embodiment of the gas measuring device being presented. For example, a possible configuration of the tester being presented can be connected for this purpose to a gas sensor to obtain a possible configuration of the gas measuring device being presented.

At least one gas generator of the gas measuring device is actuated in an actuation step 503 such that it admits the at least one test gas into the enclosing sensor chamber of the gas sensor in a first time period of a test and in a second time period of the test via the gas duct device.

In a determination step 505, which takes place simultaneously with the actuation step 503 at least from time to time, measured values determined by the gas sensor during the first time period and/or during the second time period are determined. This can advantageously be carried out by a computing unit of the gas measuring device reading out respective measured values determined by the gas sensor and storing them in a working memory.

The gas measuring device is marked in a marking step 507 as being defective if the analysis logarithms determine a deviation from the desired state. The marking takes place according to the present invention by a determination and output of test information during the first time period and/or during the second time period, the test information indicating whether the gas measuring device is defective, and wherein the determination of the test information is based on a comparison between the measured value and a predefined gas sensor threshold value or between a determined measured value curve parameter and a predefined curve parameter.

While specific embodiments of the invention have been shown and described in detail to illustrate the application of the principles of the invention, it will be understood that the invention may be embodied otherwise without departing from such principles.

### LIST OF REFERENCE NUMBERS

100, 400 Gas measuring device
101 Testing unit
103, 417 Gas sensor
105 Gas generator
107 first generator chamber
109 additional chamber
111, 401 First gas duct
113, 405 Second gas duct
115 First chamber
117 Additional chamber
119 Opening area
121 Inlet area
123 Flow guiding device
125 First computing unit
127 Second computing unit
129 First gas discharge opening
131 Additional gas discharge opening
200, 220, 300 Diagram
201, 221, 301 Ordinate
203, 223, 303 Abscissa
205, 207, 225 Curve
227, 305, 307
403 First gas generator
407 Additional gas generator
409 Gas transfer openings
411 Opening
413 Sensor diaphragm
415 Sensor interior space
419 Computing unit
500 Process
501, 503, 504, 507 Process step

What is claimed is:

1. A gas measuring device comprising:
a chemical gas sensor comprising:
a sensor chamber, the gas sensor being configured to react an analyte enclosed within the sensor chamber; and
an opening area, which forms a gas-permeable connection between the sensor chamber and an environment of the gas measuring device; and
a testing unit comprising:
a gas duct arrangement; and
a gas generator configured to send test gas into the sensor chamber of the gas sensor in a first time period of a test of the testing unit and in a second time period of the test via the gas duct arrangement, wherein:
a transportation of test gas has a different transportation characteristic during the first time period than during the second time period; and
the different transportation characteristic is based on a gas duct structure of the gas duct arrangement, or is based on a property of the test gas transported, or is based both on a gas duct structure of the gas duct arrangement and a property of the test gas transported.

2. A gas measuring device in accordance with claim 1, wherein:
the gas duct arrangement comprises a first gas duct and a second gas duct;
test gas is sent to the sensor chamber via the first gas duct during the first time period; and
test gas is sent to the sensor chamber via the second gas duct during the second time period.

3. A gas measuring device in accordance with claim 2, wherein:
the first gas duct has a first gas duct structure;
the second gas duct has a second gas duct structure; and
the first gas duct structure differs from the second gas duct structure.

4. A gas measuring device in accordance with claim 3, wherein a first gas duct opening of the first gas duct is located closer to the gas sensor than a second gas duct opening of the second gas duct.

5. A gas measuring device in accordance with claim 3, wherein the second gas duct opening is located closer to the at least one opening area than the first gas duct opening.

6. A gas measuring device in accordance with claim 3, wherein the first gas duct structure has an inner surface with a roughness that is different from a roughness of an inner surface of the second gas duct structure.

7. A gas measuring device in accordance with claim 1, wherein:
the gas duct arrangement comprises a first gas duct and a second gas duct; and
one of the gas ducts further comprises a temperature control unit configured to change a temperature in an interior of the one of the gas ducts such that a gas temperature of test gas in the first time period differs from a gas temperature of test gas in the second time period.

8. A gas measuring device in accordance with claim 1, wherein the gas generator is configured to send a different test gas through the gas duct structure during the first time period than during the second time period.

9. A gas measuring device in accordance with claim 8, wherein:
the test gas comprises a first test gas sent during the first time period and a second test gas sent during the second time period; and
the first test gas has a different diffusion coefficient than the second test gas.

10. A gas measuring device in accordance with claim 1, wherein the testing unit provides a test comprising the transportation of the test during the first time period and the second time period and further provides at least one additional time period during the test.

11. A gas measuring device in accordance with claim 1, wherein:
the gas measuring device further comprises a computing unit configured to receive a signal of the gas sensor and to determine a measured value based on the signal;
the computing unit is further configured to determine and to output test information during the first time period or during the second time period or during both the first time period and the second time period;
the test information indicates whether the gas measuring device is defective; and
the determination of the test information is based on a comparison between the measured value and a predefined gas sensor threshold value or between a determined measured value relationship parameter and a predefined relationship parameter.

12. A gas measuring device in accordance with claim 11, wherein the test information indicates whether the at least one opening area is clogged or whether the gas sensor is operable;
or both whether the at least one opening area is clogged and whether the gas sensor is operable.

13. A gas measuring device in accordance with claim 11, wherein:
the corresponding comparison carried out by the computing unit depends on a determination of at least one of a decay time, a mathematical derivation, a statistical mean, a maximum and an integral; and
the determination is based on the measured value relationship determined.

14. A tester for a gas measuring device, the tester comprising:
a testing unit comprising:
a gas duct arrangement comprising a gas duct structure; and
a gas generator configured to send test gas into a sensor chamber of a gas sensor in a first time period of a test of the testing unit and in a second time period of the test of the testing unit via the gas duct arrangement, wherein:
a transportation of test gas has a different transportation characteristic during the first time period than during the second time period; and
the different transportation characteristic is based on the gas duct structure, or is based on a property of the test gas transported, or is based both on the gas duct structure and a property of the test gas transported; and
a connection interface configured to provided a reversible operative connection with the gas sensor.

15. A tester in accordance with claim 14, wherein:
the gas duct arrangement comprises a first gas duct and a second gas duct;
test gas is sent via the first gas duct during the first time period; and
test gas is sent via the second gas duct during the second time period.

16. A tester in accordance with claim 15, wherein:
the first gas duct has a first gas duct structure;
the second gas duct has a second gas duct structure; and
the first gas duct structure differs from the second gas duct structure.

17. A tester in accordance with claim 14, wherein:
the gas duct arrangement comprises a first gas duct and a second gas duct; and
one of the gas ducts further comprises a temperature control unit configured to change a temperature in an interior of the one of the gas ducts such that a gas temperature of test gas in the first time period differs from a gas temperature of test gas in the second time period.

18. A tester in accordance with claim 14, wherein the gas generator is configured to send a different test gas through the gas duct arrangement during the first time period than during the second time period.

19. A tester in accordance with claim 18, wherein:
the test gas comprises a first test gas sent during the first time period and a second test gas sent during the second time period; and
the first test gas has a different diffusion coefficient than the second test gas.

20. A testing process for testing a gas measuring device, wherein the testing process comprises the steps of:
providing a gas measuring device comprising: a chemical gas sensor comprising: a sensor chamber, the sensor reacting an analyte enclosed within the sensor chamber; and an opening area, which forms a gas-permeable connection between the sensor chamber and an environment of the gas measuring device; and a testing unit comprising: a gas duct arrangement; and a gas generator configured to send test gas into the sensor chamber of the gas sensor in a first time period of a test of the testing unit and in a second time period of the test via the gas duct arrangement, wherein: a transportation of test gas has a different transportation characteristic during the first time period than during the second time period; and the different transportation characteristic is based on a gas duct structure of the gas duct arrangement, or is based on a property of the test gas transported, or is based both on a gas duct structure of the gas duct arrangement and a property of the test gas transported;
actuating the gas generator such that the gas generator sends test gas into the enclosing sensor chamber of the gas sensor via the gas duct arrangement during a first time period of a test and during a second time period of the test;
determining measured values during the first time period, or determining measured values during the second time period or determining measured values during both the first time period and the second time period; and
determining test information and outputting test information during the first time period or during the second time period or during both the first time period and the second time period, wherein:
the test information indicates whether the gas measuring device is defective; and
the determination of the test information is based on a comparison between the measured value or a parameter calculated therefrom and a predefined gas sensor threshold value or between a determined measured value relationship parameter and a predefined relationship parameter.
